(19) United States
(12) Patent Application Publication
(10) Pub. No.: US 2003/0148476 A1
(43) Pub. Date: Aug. 7, 2003

(54) Nucleotide sequences that code for the rplK gene and methods of use thereof

(76) Inventors: Lutz Wehmeier, Frankfurt (DE); Andreas Tauch, Bielefeld (DE); Alfred Puhler, Bielefeld (DE); Jorn Kalinowski, Bielefeld (DE); Bettina Mockel, Bielefeld (DE)

Correspondence Address: SMITH, GAMBRELL & RUSSELL, LLP, 1850 M STREET, N.W., SUITE 800, WASHINGTON, DC, 20036, US

(21) Appl. No.: 10/302,931

(22) Filed: Nov. 25, 2002

Related U.S. Application Data
(62) Division of application No. 09/568,023, filed on May 10, 2000.

(51) Int. Cl. C12P 13/08; C07H 21/04; C12N 9/10; C12N 15/74; C12P 21/02; C12N 1/21

(52) U.S. Cl. 435/115; 435/69.1; 435/193; 435/252.3; 435/320.1; 536/23.2

(57) ABSTRACT

An isolated polynucleotide from coryneform bacteria containing a polynucleotide sequence selected from the group:

a) a polynucleotide that is at least 70% identical to a polynucleotide that codes for a polypeptide that contains the amino acid sequence of SEQ ID NO: 2,

b) a polynucleotide that codes for a polypeptide that contains an amino acid sequence that is at least 70% identical to the amino acid sequence of SEQ ID NO: 2,

c) a polynucleotide that is complementary to the polynucleotides of (a) or (b), and

d) a polynucleotide containing at least 15 successive bases of the polynucleotide sequence of (a), (b) or (c),

and methods of use thereof.

The object of the invention resides in nucleotide sequences from coryneform bacteria that code for the rplK gene and a method for enzymatic preparation of amino acids, especially L-lysine, while attenuating the rplK gene.

All references cited herein are incorporated by reference into this specification. In addition, throughout the following disclosure, the term “incorporated by reference” is indicated by the notation “I.B.R.”

### BACKGROUND OF THE INVENTION

L-Amino acids, especially L-lysine, are used in animal nutrition, in human medicine and in the pharmaceutical industry. It is known that these amino acids are prepared by fermentation of strains of coryneform bacteria, especially Corynebacterium glutamicum. Because of their great importance, work to improve the production methods is continuously being carried out. Methods of mutagenesis, selection and mutant selection are used to improve the performance properties of these microorganisms. In this way one obtains strains that are resistant to antimetabolites such as the lysine analog S-(2-aminoethyl)cysteine or auxotrophic for regulatorily important metabolites and produce L-amino acids.

For a number of years methods of recombinant DNA technology have also been used for strain improvement of strains of Corynebacterium that produce L-amino acid, by amplifying individual amino acid biosynthesis genes and testing the effect on L-amino acid production. Review articles on this subject can be found, among other places, in Kinoshita (“Glutamic Acid Bacteria,” in: Biology of Industrial Microorganisms, Demain and Solomon (Eds.), Benjamin Cummings, London, UK, 1985, 115-142) I.B.R., Hilliger (BioTec 2, 40-44 (1991) I.B.R.), Eggeling (Amino Acids 6:261-272 (1994) I.B.R.), Jetten and Sinskey (Critical Reviews in Biotechnology 15, 73-103 (1995) I.B.R.) and Sahm et al. (Annuals of the New York Academy of Science 782, 25-39 (1996) I.B.R.).

The rplK protein (ribosomal large subunit protein K) is a component of the bacterial ribosome that was first described for Escherichia coli, the translation apparatus of the cell, on which protein synthesis takes place.

Ribosomes are cellular particles that are composed of three ribonucleic acid (RNA) molecules and a specific number of proteins. Ribosomes are for the most part obtained from cell extracts by ultracentrifugation. The further purification of the remaining cell components usually takes place by sedimentation in sucrose gradients. This preparation technique led to the usual designations for the components of ribosomes, which refer directly to the sedimentation properties. Thus, one functional bacterial ribosome is frequently designated 70S ribosome, which consists of the small (small subunit) 30S subunit and the large (large subunit) 50S subunit. The small 30S subunit of E. coli consists of 21 different polypeptides and an RNA molecule with a length of 1542 nucleotides, which is known to the specialist as 16S-rRNA; besides the rplK protein, the large 50S subunit contains an additional 31 different polypeptides, together with two RNA molecules with lengths of 120 and 2904 nucleotides, respectively, the so called 5S or 23S rRNA molecules. Meanwhile, an alternative nomenclature has been established for the ribosomal proteins. Thus, the polypeptides of the small 30S subunit are designated S1 to S21 by the specialist, while those of the large 50S subunit are designated L1 to L32. The rplK protein here corresponds to the ribosomal L11 protein (Noller and Nomura, In: Neidhardt et al., Escherichia coli and Salmonella typhimurium: Cellular and molecular biology. American Society for Microbiology, Washington D.C., 167-186, 1996) I.B.R. In recent years L11-like proteins have also been identified in other organisms such as Borrelia burgdorferi (Fraser et al., Nature, 390, 580-586, 1997) I.B.R., Helicobacter pylori (Tomb et al., Nature, 388, 539-547, 1997) I.B.R., Serratia marcescens (Sor and Nomura, Molecular and General Genetics, 210, 52-59, 1987) I.B.R., Haemophilus infuenzae (Fleischmann et al., Science, 269, 496-512, 1995) I.B.R. and in the gram positive bacterium Bacillus subtilis (Jeong et al., Molecular Microbiology, 10, 133-142, 1993) I.B.R.

The process of translation that occurs at the ribosome, thus the messenger RNA-controlled biosynthesis of polypeptides, is complex. In addition to the ribosome, other proteins called protein synthesis factors by the specialist (Noller, Annual Review of Biochemistry, 60, 191-227, 1991) I.B.R., are essential for the translation process. The L11 protein mediates the interaction between the ribosome and some protein synthesis factors; one may mention as an example here the elongation factor G (EF-G) and the termination factor 1 (RF-1). The absence of the L11 protein in the ribosomes of E. coli L11 mutants thus leads to a reduction of the translation rate (Xing and Draper, Biochemistry, 35, 1581-1588, 1996) I.B.R.

The L11 protein is likewise essential for the bonding and activity of the RelA protein to the ribosome. RelA catalyzes, under conditions where there is a deficiency of amino acid, the synthesis of guanosine tetraphosphate (ppGpp) by the transfer of one pyrophosphate group from ATP to GDP. In E. coli ppGpp affects the expression of many genes, either negatively or positively. In general, the expression of gene products that are effective in biosynthesis pathways is stimulated. Gene products that are catabolically effective are as a rule correspondingly negatively regulated. A large number of genes and operons that play a central role in amino acid biosynthesis are regulated by ppGpp in E. coli. Among the genes known up to now to be positively affected in E. coli are argF, argI, argECBH (arginine biosynthesis), gltB, glnA, gdh (glutamine/glutamate biosynthesis), ilvB, IlvA (isoleucine biosynthesis), metC, metF, metK (methionine biosynthesis), thrA, thrB, thrC (threonine biosynthesis), lysA, lysC, dapB, asd (lysine biosynthesis) (Cashel et al., In: Neidhardt et al., Escherichia coli and Salmonella typhimurium: Cellular and molecular biology, American Society for Microbiology, Washington D.C., 1458-1496, 1996) I.B.R. Meanwhile, the function of ppGpp as a positive regulator of amino acid biosynthesis was also demonstrated in other bacteria such as Salmonella typhimurium (Rudd et al., Journal of Bacteriology, 163, 534-542, 1985) I.B.R., Vibrio sp. (Flärdh et al., Journal of Bacteriology, 176, 5949-5957, 1994) I.B.R. and B. subtilis (Wendrich and Marahiel, Molecular Microbiology, 26, 65-79, 1997) I.B.R.

### OBJECTIVE OF THE INVENTION

From the prior art it is clear that there is interest in finding out if knowledge of the nucleotide sequence of the rplK gene of coryneform bacteria will contribute to an improvement of the amino acid production of these bacteria. Thus, an object of the invention is to make available new measures for improved enzymatic preparation of amino acids, especially L-lysine.

### SUMMARY OF THE INVENTION

L-Amino acids, especially lysine, are used in human medicine and in the pharmaceutical industry, in the food industry and particularly in animal nutrition. For this reason there is general interest in making available new approaches for improved methods for preparation of amino acids, especially L-lysine.

A feature of the invention is an isolated polynucleotide containing a polynucleotide sequence chosen from the group

a) a polynucleotide that is at least 70% identical to a polynucleotide that codes for a polypeptide that contains the amino acid sequence of SEQ ID No. 2,

b) a polynucleotide that codes for a polypeptide that contains an amino acid sequence that is at least 70% identical to the amino acid sequence of SEQ ID No. 2.

The relative degree of substitution or mutation in the polynucleotide or amino acid sequence to produce this percentage of sequence identity can be established or determined by well-known methods of sequence analysis. These methods are disclosed and demonstrated in Bishop, et al. “DNA & Protein Sequence Analysis (A Practical Approach”), Oxford Univ. Press, Inc. (1997) I.B.R. and by Steinberg, Michael “Protein Structure Prediction” (A Practical Approach), Oxford Univ. Press, Inc. (1997) I.B.R.,

c) a polynucleotide that is complementary to the polynucleotides of (a) or (b), and

d) a polynucleotide containing at least 15 successive bases of the polynucleotide sequence of (a), (b) or (c).

Another feature of the invention is the polynucleotide in accordance with (a-d) above, where it is preferably a replicable DNA containing:

(i) the nucleotide sequence indicated in SEQ ID No. 1, or

(ii) at least one sequence that corresponds to the sequence (i) within the region of degeneration of the genetic code, or

(iii) at least one sequence that hybridizes with the sequence complementary to sequence (i) or (ii). The degree of stringency required to produce hybridization may vary from high to low as described in Sambrook et al. and other documents incorporated by reference herein and optionally

(iv) function-neutral sense mutants in (i).

Other features of the invention include:

a polynucleotide which is replicable, preferably recombinant DNA, containing the nucleotide sequence as represented in SEQ ID No. 1,

a polynucleotide which is replicable, preferably recombinant DNA, which codes for a polypeptide that contains the amino acid sequence as represented in SEQ ID No. 2,

a polynucleotide as in (a-d) above, especially item (d), containing the nucleotide sequence as represented in SEQ ID No. 3,

a polynucleotide as in (a-d) above, especially item (d), which codes for a polypeptide that contains the amino acid sequence as represented in SEQ ID No. 4,

a vector containing a mutated polynucleotide as in (a-d) above, especially item (d) represented in SEQ ID No. 3 and FIG. 1 (Δ=deHa) and deposited in E. coli DH5α/pΔrplK as DSM 13158 in accordance with the Budapest Treaty and deposited on Nov. 26, 1999 with the International Depositary Authority of DSMZ-Qeutsche Sammlung von Mikroorganism Und Zell Kulturen GmbH, Maschroder Weg 1b, D-38124 Braunschweig, Germany,

and coryneform bacteria serving as host cells, which contain an insertion or deletion in the rplK gene.

A further feature of the invention is also polynucleotides that essentially consist of a polynucleotide sequence that can be obtained by screening by means of hybridization, with varying degrees of stringency as established in Sambrook and the other citations incorporated by reference herein, of an appropriate gene bank that contains the complete gene with the polynucleotide sequence in correspondence with SEQ ID No. 1, with a probe that contains the sequence of said polynucleotide in accordance with SEQ ID No. 1 or a fragment thereof, and isolation of the said DNA sequence.

Polynucleotide sequences in accordance with the invention are suitable as hybridization probes for RNA, cDNA and DNA, in order to isolate cDNA in its full length, that code for the ribosomal protein L11 and to isolate those cDNA or genes that have high similarity to the sequence with the rplK gene.

Polynucleotide sequences in accordance with the invention are also suitable as primers, with which the polymerase chain reaction (PCR) of the DNA of genes that code for the rplK gene product or ribosomal protein L11 can be produced.

Oligonucleotides that serve as probes or primers contain at least 30, preferably at least 20, really especially preferably at least 15 successive nucleotides. Likewise suitable are oligonucleotides with a length of at least 40 or 50 base pairs.

“Isolated” means separated from its natural environment.

“Polynucleotide” refers in general to polyribonucleotides and polydeoxyribonucleotides, where these can be unmodified RNA or DNA or modified RNA or DNA.

“Polypeptides” is understood to mean peptides or proteins that contain two or more amino acids bonded via peptide linkages.

The polypeptides in accordance with the invention include the polypeptide in accordance with SEQ ID No. 2, especially ones with the biological activity of the rplK gene product and also those that are at least 70% identical to the polypeptide in accordance with SEQ ID No. 2, preferably at least 80% and especially at least 90 to 95% identical to the polypeptide in accordance with SEQ ID No. 2 and have the same activity. The degree of substitution or mutation in the polynucleotide or amino acid sequence to produce this degree of identity can be determined by well-known methods of sequence analysis. These methods are disclosed and demonstrated in Bishop, et al. “DNA & Protein Sequence Analysis (A Practical Approach”), Oxford Univ. Press, Inc. (1997) I.B.R. and by Steinberg, Michael “Protein Structure Prediction” (A Practical Approach), Oxford Univ. Press, Inc. (1997) I.B.R.

The invention additionally concerns a method for enzymatic preparation of amino acids, especially lysine, while using coryneform bacteria that in particular already produce the amino acids and in which the nucleotide sequences that code for the rplK gene have been attenuated.

The term “attenuation” in this connection describes the reduction or switching off of the intracellular activity or function of one or more enzymes or proteins in a microorganism that are coded by the corresponding DNA, by using, for example, a weak promoter or a gene or allele that codes for a corresponding enzyme or protein with low activity or that inactivates the corresponding gene or enzyme or protein and optionally by combining these measures.

The microorganisms that are objects of this invention can produce amino acids, especially lysine, from glucose, sucrose, lactose, fructose, maltose, molasses, starch, cellulose or from glycerol and ethanol. These can be representatives of coryneform bacteria, especially the genus Corynebacterium. In the genus Corynebacterium one should especially mention the species Corynebacterium glutamicum, which is known among specialists for its capacity to produce L-amino acids.

### BRIEF DESCRIPTION OF THE DRAWING

The present invention will be further understood with reference to the drawing offered here for illustration only and not in limitation of this invention.

In the drawing:

FIG. 1 is a map of the plasmid pΔrplK.

The abbreviations and designations that are used have the following meanings:

| | |
|---|---|
| pdeltarplK | pΔrplK |
| sacB gene | sacB gene from Bacillus subtilis, coded for the enzyme |
| | levansucrase |
| lacZ alpha: | Part of the 5′ end of the 13-galactosidase gene |
| oriV | Replication origin |
| KmR: | Kanamycin resistance |
| RP4 mob | mob region of plasmid RP4 |
| BamHI: | Scission site of the restriction enzyme BamHI |
| EcoRI: | Scission site of the restriction enzyme EcoRI |
| ΔrplK | rplK allele with a deletion of 12 pb in the N-terminal region |

### BRIEF DESCRIPTION OF SEQUENCE DATA

SEQ ID NO 1 is a new nucleotide sequence that codes for the rplK gene.

SEQ ID NO 2 is the amino acid sequence of the rplK gene product (the L-11 gene).

SEQ ID NO 3 is an allele of the rplK gene.

SEQ ID NO 4 is a variation of protein L-11 coded by the ΔrplK allele.

SEQ ID NO 5 is a P1 up primer derived on the basis of SEQ ID NO 1.

SEQ ID NO 6 is a P2 down primer derived on the basis of SEQ ID NO 1.

SEQ ID NO 7 is a P1 up primer derived on the basis of SEQ ID NO 1.

SEQ ID NO 8 is a P2 down primer derived on the basis of SEQ ID NO 1.

### DETAILED DESCRIPTION OF THE INVENTION

Suitable strains of genus Corynebacterium, especially the species Corynebacterium glutamicum, are in particular the known wild strains

Corynebacterium glutamicum ATCC13032

Corynebacterium acetoglutamicum ATCC15806

Corynebacterium acetoacidophilum ATCC 13870

Corynebacterium melassecola ATCC17965

Corynebacterium thermoaminogenes FERM BP-1539

Brevibacterium flavum ATCC 14067

Brevibacterium lactofermentum ATCC13869 and

Brevibacterium divaricatum ATCC14020

and mutants or strains that produce L-amino acids and are prepared from them, such as, for example, the L-lysine producing strains

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464

Corynebacterium glutamicum DSM 5715

Corynebacterium glutamicum DSM 12866 and

Corynebacterium glutamicum DM58-1.

The inventors were successful in isolating the new rplK gene from Corynebacterium glutamicum.

For isolation of the rplK gene of C. glutamicum a gene bank of Corynebacterium glutamicum is first constructed. The formation of gene banks is described in generally known textbooks and manuals. Examples that may be mentioned include the textbook by Winnacker: Genes and Clones: an Introduction to Gene Technology (Verlag Chemie, Weinheim, Germany, 1990) I.B.R. or the manual by Sambrook et al.: Molecular Cloning. A Laboratory Manual (Cold Spring Harbor Laboratory Press, 1989) I.B.R. A very well known gene bank is that of E. coli K-12 strain W3110, which was designed by Kohara et al. (Cell 50, 495-508 (1987) I.B.R.) in λ-vectors. Bathe et al. (Molecular and General Genetics, 252:255-265, 1996) I.B.R. describe a gene bank of C. glutamicum ATCC13032, which was constructed with the aid of the cosmid vector SuperCos I (Wahl et al., 1987, Proceedings of the National Academy of Sciences USA, 84:2160-2164) I.B.R. in the E. coli K-12 strain NM554 (Raleigh et al., 1988, Nucleic Acids Research 16:1563-1575) I.B.R. Börmann et al. (Molecular Microbiology 6(3), 317-326) I.B.R. again describe a gene bank of C. glutamicum ATCC13032 using the cosmid pHC79 (Hohn and Collins, Gene 11, 291-298 (1980) I.B.R.

Plasmids like pBR322 (Bolivar, Life Sciences, 25, 807-818 (1979) I.B.R.) or pUC9 (Vieira et al., 1982, Gene, 19:259-268) I.B.R. can also be used to prepare a gene bank of C. glutamicum in E. coli. Strains of E. coli that are restriction and recombination defective are especially suitable as hosts. An example of this is the strain DH5αmcr, which was described by Grant et al. (Proceedings of the National Academy of Sciences USA, 87 (1990) 4645-4649) I.B.R.

The long DNA fragments cloned with the aid of cosmids can finally again be subcloned into common vectors that are suitable for sequencing.

Methods for DNA sequencing are described, among other places, in Sanger et al. (Proceedings of the National Academy of Sciences of the United States of America USA, 74:5463-5467, 1977) I.B.R.

The new DNA sequence of C. glutamicum that codes for the rplK gene, which as SEQ ID NO 1 is the object of this invention, is obtained in this way. In addition, the amino acid sequence of the corresponding protein was derived from this DNA sequence with the above described methods. The resulting amino acid sequence of the rplK gene product or the L-11 gene is represented in SEQ ID NO 2.

The coding DNA sequences that result from SEQ ID NO 1 through the degeneracy of the genetic code are likewise objects of the invention. In the same way, DNA sequences that hybridize with SEQ ID NO 1 or parts of SEQ ID NO 1 are objects of the invention. Hybridization can occur at varying degrees of stringency. Amino acid sequences that are obtained in the corresponding way from SEQ ID NO 1 are likewise objects of the invention.

The invention has found that coryneform bacteria, after attenuation of the rplK gene, produce L-amino acids, especially L-lysine, in an improved way.

To achieve an attenuation, either the expression of the rplK gene or preferably the functional properties of the protein can be reduced. Optionally the two measures can be combined.

Reduction of gene expression can take place by suitable culturing or through genetic alteration (mutation) of the signal structures of gene expression. Signal structures of gene expression are, for example, repressor genes, activator genes, operators, promoters, attenuators, ribosome binding sites, the start codons and terminators. The specialist will find information in this regard, for example, in Patent Application WO 96/15246 I.B.R., in Boyd and Murphy (Journal of Bacteriology 170: 5949 (1988) I.B.R.), in Voskuil and Chambliss (Nucleic Acids Research 26: 3548 (1998) I.B.R.), in Jensen and Hammer (Biotechnology and Bioengineering 58: 191 (1998) I.B.R.), in Patek et al. (Microbiology 142: 1297 (1996) I.B.R.) and in well known textbooks on genetics and microbiology such as, for example, the textbook by Knippers (Molecular Genetics, 6^{th} edition, Georg Thieme Verlag, Stuttgart, Germany, 1995) I.B.R. or the one by Winnacker (Genes and Clones, VCH Verlagsgesellschaft, Weinheim, Germany, 1990) I.B.R.

Mutations that lead to an alteration or reduction of the functional properties of proteins are known from the prior art. One may mention as examples the works by Qiu and Goodman (Journal of Biological Chemistry 272: 8611-8617 (1997) I.B.R.), Sugimoto et al. (Bioscience Biotechnology and Biochemistry 61: 1760-1762 (1997) I.B.R.) and Möckel (“Threonine dehydratase from Corynebacterium glutamicum: elimination of allosteric regulation and the structure of the enzyme,” Reports of the Jülich Research Center, Jül-2906, ISSN09442952, Jülich, Germany, 1994) I.B.R. Summarizations can be taken from well known textbooks on genetics and molecular biology such as the one by Hagemann (General Genetics, Gustav Fischer Verlag, Stuttgart, 1986) I.B.R.

Transitions, transversions, insertions and deletions are possibilities as mutations. One speaks of missense mutations or nonsense mutations, depending on the effect of the amino acid exchange on activity. Insertions or deletions of at least one base pair in a gene lead to frame shift mutations, as a consequence of which wrong amino acids are incorporated or the translation is prematurely cut short. Instructions for producing such mutations belong to the prior art and can be taken from well known textbooks on genetics and molecular biology such as the textbook by Knippers (Molecular Genetics, 6^{th} edition, Georg Thieme Verlag, Stuttgart, Germany, 1995) I.B.R., the one by Winnacker (Genes and Clones, VCH Verlagsgesellschaft, Weinheim, Germany, 1990) I.B.R. or by Hagemann (General Genetics, Gustav Fischer Verlag, Stuttgart, 1986) I.B.R. Methods for producing mutations with the aid of the polymerase chain reaction (PCR) are described in C. R. Newton and A. Graham, PCR, 2^{nd} edition, Spektrum Akademischer Verlag, Heidelberg, 1997, I.B.R.

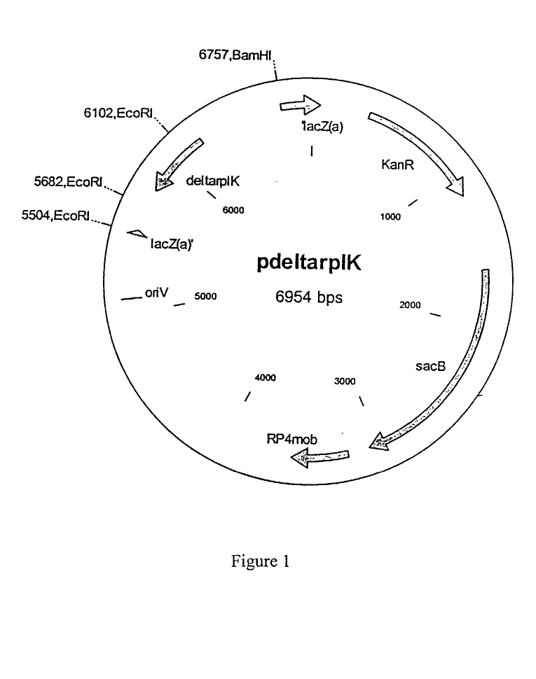

An example of a plasmid with which a deletion mutagenesis of the rplK gene can be carried out is the plasmid pΔrplK represented in FIG. 1.

Plasmid pΔrplK consists of the plasmid pK18mobsacB described by Jäger et al. (Journal of Bacteriology, 1992, 174: 5462-65) I.B.R., in which an allele of the rplK gene, represented in SEQ ID No. 3, was inserted. The allele designated ΔrplK carries a 12 bp-long deletion in the 5′ region of the gene. The variation of protein L11 coded by the ΔrplK allele is represented as SEQ ID No. 4. The variation of protein L-11 as represented differs from the wild type of protein L11 by the error in the tetrapeptide proline-alanine-leucine-glycine corresponding to the amino acids of position 30 to 33 of SEQ ID No. 2.

Plasmid pΔrplK leads to exchange of the chromosomal rplK gene for the ΔrplK allele after homologous recombination. Instructions and illustrations for insertion mutagenesis can be found, for example, in Schwarzer and Pühler (Bio/Technology 9, 84-87 (1991) I.B.R.) or Fitzpatrick et al. (Applied Microbiology and Biotechnology 42, 575-580 (1994) I.B.R.).

In addition, it can be advantageous for the production of L-amino acids, especially L-lysine, to enhance one or more enzymes of the relevant biosynthesis path, especially to overexpress them in addition to attenuation of the rplK gene.

For example, especially for the preparation of L-lysine, one or more of the genes chosen from the group

the dapA gene coding for dihydrodipicolinate synthase (EP-B 0 197 335) I.B.R.,

an lysC gene coding for a feedback resistant aspartate kinase (Kalinowski et al. (1990) I.B.R., Molecular and General Genetics 224: 317-324) I.B.R.,

the pyc gene coding for pyruvate carboxylase (Eikmanns (1992), Journal of Bacteriology 174:6076-6086) I.B.R.,

the mqo gene coding for malate quinone oxidoreductase (Molenaar et al., European Journal of Biochemistry 254, 395-403 (1998) I.B.R.),

the lysE gene coding for lysine export (DE-A-195 48 222) I.B.R.,

the zwa1 gene (DE 199 59 328.0; DSM 13115) I.B.R.,

for example, can be simultaneously enhanced, especially overexpressed.

In addition, it can be advantageous for the production of L-amino acids, in addition to attenuation of the rplK gene, to attenuate at the same time one or more of the genes chosen from the group:

the pck gene coding for phosphoenol pyruvate carboxykinase (DE 199 50 409.1; DSM 13047) I.B.R.,

the pgi gene coding for glucose 6-phosphate isomerase (U.S. Ser. No. 09/396,478, DSM 12969) I.B.R.,

the poxB gene coding for pyruvate oxidase (DE 199 51 975.7; DSM 13114) I.B.R.,

the zwa2 gene (DE: 199 59 327.2; DSM 13113) I.B.R.,

the relA gene coding for PPGPP synthetase I (EC 2.7.6.5) I.B.R.

In addition, it can be advantageous for the production of L-amino acids, in addition to the overexpression of 6-phosphogluconate dehydrogenase, to switch off undesired side reactions (Nakayama: “Breeding of Amino Acid Producing Micro-organisms,” in: Overproduction of Microbial Products, Krumphanzl, Sikyta, Vanek (eds.), Academic Press, London, UK, 1982) I.B.R.

The microorganisms containing the mutated polynucleotide in accordance with (a-d) above, particularly item (d), are likewise objects of the invention and can be cultured continuously or batchwise in a batch process or in a fed batch process or a repeated fed batch process for purposes of producing L-amino acids, especially L-lysine. A summary of the known cultivation methods is described in the textbook by Chmiel (Bioprocess Engineering. 1. Introduction to Bioprocess Techniques (Gustav Fischer Verlag, Stuttgart, 1991) I.B.R.) or in the textbook by Storhas (Bioreactors and Peripheral Equipment (Vieweg Verlag, Braunschweig/Wiesbaden, 1994) I.B.R.).

The culture medium that is to be used must satisfy the requirements of the relevant strain in a suitable way. Descriptions of culture media for various microorganisms can be found in the manual “Manual of Methods for General Bacteriology,” of the American Society for Bacteriology (Washington, D.C., USA, 1981) I.B.R. Sugar and carbohydrates such as glucose, sucrose, lactose, fructose, maltose, molasses, starch and cellulose, oils and fats like soy oil, sunflower seed oil, peanut oil and coconut oil, fatty acids such as palmitic acid, stearic acid and linolic acid, alcohols such as glycerol and ethanol and organic acids like acetic acid can be used as carbon sources. These substances can be used individually or as a mixture. Compounds like peptone, yeast extract, meat extract, malt extract, corn steep liquor, soy flour and urea or inorganic compounds like ammonium sulfate, ammonium chloride, ammonium phosphate, ammonium carbonate and ammonium nitrate can be used as sources of nitrogen. The nitrogen sources can be used individually or as a mixture. Phosphoric acid, potassium dihydrogen phosphate or dipotassium hydrogen phosphate or the corresponding sodium-containing salts can be used as phosphorus sources. The culture medium must additionally contain salts of metals such as magnesium sulfate or iron sulfate, which are necessary for growth. Finally, essential growth-promoter substances like amino acids and vitamins can be used in addition to the substances mentioned above. Suitable precursors can also be added to the culture medium. Said substances can be added to the culture in the form of a single batch or can be dispensed during cultivation in a suitable way.

Basic compounds like sodium hydroxide, potassium hydroxide, ammonia or ammonia water or acid compounds like phosphoric acid or sulfuric acid are used as appropriate to control the pH of the culture. Antifoam agents like fatty acid polyglycol esters can be used to control the formation of foam. To maintain the stability of plasmids, suitable selectively active substances such as antibiotics can be added to the medium. In order to maintain aerobic conditions, oxygen or oxygen-containing gas mixtures such as air can be introduced into the culture. The temperature of the culture is normally 20° C. to 45° C. and preferably 25° C. to 40° C. The culture is continued until a maximum of the desired product has formed. This goal is normally achieved in a period of 10 h to 160 h.

Methods for determining L-amino acids are known from the prior art. The analysis can be carried out as described in Spackman et al. (Analytical Chemistry, 30, (1958), 1190) I.B.R. by anion exchange chromatography followed by ninhydrin derivatization, or it can be carried out by reversed phase HPLC, as described in Lindroth et al. (Analytical Chemistry (1979) 51: 1167-1174) I.B.R.

The following microorganism was added to the German Collection of Microorganisms and Cell Cultures (DSMZ, Braunschweig, Germany) in accordance with the Budapest Treaty:

Escherichia coli strain DH5α/pΔrplK, as DSM 13158.

### EXAMPLES

### Example 1

Preparation of a Genomic Cosmid Gene Bank from Corynebacterium glutamicum ATCC 13032

Chromsomal DNA was isolated from Corynebacterium glutamicum ATCC 13032 as described in Tauch et al. (1995, Plasmid 33:168-179) I.B.R. and partially cleaved with the restriction enzyme Sau3AI (Amersham Pharmacia, Freiburg, Germany, product description Sau3AI, Code No. 27-0913-02) I.B.R. The DNA fragments were dephophosphorylated with shrimp alkaline phosphatase (Roche Molecular Biochemicals, Mannheim, Germany, product description SAP, Code No. 1758250) I.B.R. The DNA of the cosmid vector SuperCos1 (Wahl et al. (1987) I.B.R. Proceedings of the National Academy of Sciences USA 84:2160-2164) I.B.R., purchased from the Stratagene Company (La Jolla, USA, product description SuperCos1 cosmid Vector Kit, Code No. 251301) I.B.R., was cleaved with the restriction enzyme XbaI (Amersham Pharmacia, Freiburg, Germany, product description XbaI, Code No. 27-0948-02) I.B.R. and likewise dephosphorylated with shrimp alkaline phosphatase. Then the cosmid DNA was cleaved with the restriction enzyme BamHI (Amersham Pharmacia, Freiburg, Germany, product description BamHI, Code No. 27-0868-04) I.B.R. The cosmid DNA treated in this way was mixed with the treated ATCC13032 DNA and the batch was treated with T4-DNA ligase (Amersham Pharmacia, Freiburg, Germany, product description T4-DNA-ligase, Code No. 27-0870-04) I.B.R. The ligation mixture was then packaged in phages using the Gigapack II XL Packing Extracts (Stratagene, La Jolla, USA, product description Gigapack II XL Packing Extract, Code No. 200217) I.B.R. To infect the E. coli strain NM554 (Raleigh et al., 1988, Nucleic Acid Research 16:1563-1575) I.B.R., the cells were taken up in 10 mM MgSO₄ and mixed with an aliquot portion of the phage suspension. Infection and titration of the cosmid bank were carried out as described in Sambrook et al. (1989, Molecular Cloning: A Laboratory Manual, Cold Spring Harbor) I.B.R., with the cells being plated out onto LB agar (Lennox, 1955, Virology, 1:190) I.B.R. with 100 mg/L ampicillin. After incubation overnight at 37° C. recombinant single clones were selected.

### Example 2

Isolation and Sequencing of the rplK Gene

The cosmid DNA of a single colony was isolated with the Qiaprep Spin Miniprep Kit (Product No. 27106, Qiagen, Hilden, Germany) I.B.R. according to the manufacturer's instructions and partially cleaved with the restriction enzyme Sau3AI (Amersham Pharmacia, Freiburg, Germany, product description Sau3AI, Product No. 27-0913-02) I.B.R. The DNA fragments were dephosphorylated with shrimp alkaline phosphatase (Roche Molecular Biochemicals, Mannheim, Germany, product description SAP, Product No. 1758250) I.B.R. After gel electrophoretic separation the cosmid fragments in the size range of 1500 to 2000 bp were isolated with the QiaExII gel extraction kit (Product No. 20021, Qiagen, Hilden, Germany) I.B.R. The DNA of the sequencing vector pZero-1, purchased from the Invitrogen Company (Groningen, Netherlands, product description Zero Background Cloning Kit, Product No. K2500-01) I.B.R. was cleaved with the restriction enzyme BamHI (Amersham Pharmacia, Freiburg, Germany, product description BamHI, Product No. 27-0868-04) I.B.R. Ligation of the cosmid fragments in the sequencing vector pZero-1 was carried out as described by Sambrook et al. (1989, Molecular Cloning: A Laboratory Manual, Cold Spring Harbor) I.B.R., with the DNA mixture having been incubated overnight with T4 ligase (Pharmacia Biotech, Freiburg, Germany). This ligation mixture was then electroporated (Tauch et al., 1994, FEMS Microbiol Letters, 123:343-7) I.B.R. in the E. coli strain DH5αMCR (Grant, 1990, Proceedings of the National Academy of Sciences U.S.A., 87:4645-4649) I.B.R. and plated out on LB agar (Lennox, 1955, Virology, 1:190) I.B.R. with 50 mg/L zeocin. Plasmid preparation of the recombinant clones took place with the Biorobot 9600 (Product No. 900200, Qiagen, Hilden, Germany) I.B.R. Sequencing followed the dideoxy chain termination method of Sanger et al. (1977, Proceedings of the National Academies of Sciences U.S.A., 74:5463-5467) I.B.R. as modified by Zimmermann et al. (1990, Nucleic Acids Research, 18:1067) I.B.R. The “RR dRhodamin Terminator Cycle Sequencing Kit” of PE Applied Biosystems (Product No. 403044, Weiterstadt, Germany) I.B.R. was used. The gel electrophoretic separation and analysis of the sequencing reaction took place in a Rotiphorese NF acrylamide/bisacrylamide gel (29:1) (Product No. A124.1, Roth, Karlsruhe, Germany) I.B.R. with the ABI Prism 377 sequencing device of PE Applied Biosystems (Weiterstadt, Germany).

The raw sequencing data that were obtained were then processed using the Staden program package (1986, Nucleic Acids Research, 14:217-231) I.B.R. version 97-0. The single sequences of the pZero1 derivatives were assembled to a connected contig. The computer aided coding region analysis was produced with the program XNIP (Staden, 1986, Nucleic Acids Research, 14:217-231) I.B.R. Further analyses were carried out with the BLAST search programs (Altschul et al., 1997, Nucleic Acids Research, 25:3389-3402) I.B.R. against the nonredundant data bank of the National Center for Biotechnology Information (NCBI, Bethesda, Md., USA) I.B.R. in its entirety as of May 5, 2000 (including all analytical tools for sequence analysis available as of that date at that web-site).

The resulting nucleotide sequence is represented in SEQ ID NO 1. Analysis of the nucleotide sequence gave an open reading frame of 438 base pairs, which was characterized as the rplK gene. The rplK gene codes for a polypeptide of 145 amino acids, which is represented in SEQ ID No. 2.

### Example 3

Preparation of a Vector With a Copy of the rplK Gene

A chromosomal 1200 bp DNA fragment, which contained the rplK gene from C. glutamicum, was cloned by means of PCR.

For this chromosomal DNA was isolated from Corynebacterium glutamicum ATCC 13032 as described in Tauch et al. (1995, Plasmid 33:168-179) I.B.R. A 1200 bp DNA fragment that contained the rplK gene was amplified by means of the polymerase chain reaction. In addition, the following primers were derived on the basis of SEQ ID No. 1.

| | |
|---|---|
| P1 up: | (see also SEQ ID No. 5) |

5′-AGG AGC AGG CTG TTG TCA CC-3′

| | |
|---|---|
| P2 down: | (see also SEQ ID No. 6) |

5′-GCG GAT AGC TAC TGC GAT GG-3′:

The represented oligonucleotides were synthesized by the ARK Scientific Company (ARK Scientific GmbH Biosystems, Darmstadt, Germany) and the PCR reaction was carried out using the Pfu-DNA polymerase (Stratagene, La Jolla, USA) and PTC 100 thermocycler (MJ Research Inc., Waltham, USA).

A cycle consisting of thermal denaturing (94° C., 90 sec), annealing (58° C., 90 sec) and the polymerase reaction (72° C., 90 sec) was carried out 35 times in the PCR. The resulting 1200 bp DNA fragment was then purified by means of the Qiagen PCR purification spin kit (Qiagen, Hilden, Germany).

For the cloning of the DNA amplificate containing the rplK gene to a plasmid that can replicate in C. glutamicum, the plasmid pECM3, a deletion derivative of the plasmid pECM2 described in Tauch et al. (FEMS Microbiological Letters, 123, 343-347 (1994) I.B.R.) was prepared. For this the plasmid pECM2 was digested with the restriction enzymes BamHI (Amersham-Pharmacia, Freiburg, Germany) and BglII (Amersham-Pharmacia, Freiburg, Germany) and treated with T4 ligase (Amersham-Pharmacia, Freiburg, Germany), as described in Sambrook et al. (Molecular Cloning: A Laboratory Manual, Cold Spring Harbor Laboratory (1989) I.B.R.), thus producing the plasmid pECM3. Transformation of the E. coli strain DH5αMCR described in Grant et al. (Proceedings of the National Academy of Science USA, 87, 4645-4649 (1990) I.B.R.) with the plasmid pECM3 took place as described in Tauch et al. I.B.R. (FEMS Microbiological Letters, 123, 343-347 (1994) I.B.R.). The transformants were selected on LBG agar (10 g trypton, 5 g yeast extract, 5 g NaCl, 2 g glucose, 15 g agar per liter), to which chloramphenicol (Merck, Darmstadt, Germany) (50 mg/L) had been added.

Then the rplK gene-containing 1200 bp DNA amplificate was mixed with the plasmid pECM3, which had been linearized previously with the restriction enzyme SmaI (Amersham-Pharmacia, Freiburg, Germany) and treated with T4 DNA ligase (Amersham-Pharmacia, Freiburg, Germany), thus producing the plasmid prplK. Transformation of the E. coli strain DH5αMCR with the plasmid prplK took place as described in Tauch et al. (FEMS Microbiological Letters, 123, 343-347 (1994) I.B.R.), and the transformants were selected on LBG agar, to which chloramphenicol (Merck, Darmstadt, Germany) (50 mg/L) had been added. The plasmid prplK thus carries the complete rplK gene of C. glutamicum and can replicate autonomously both in E. coli and in C. glutamicum.

### Example 4

Insertion of a Deletion into the rplK Gene

An allele of the rplK gene that carries a 12 bp long deletion and is characterized as ΔrplK was prepared by means of PCR. The resulting 12 bp deletion in the rplK gene leads to loss of the tetrapeptide proline-alanine-leucine-glycine in the N-terminal region of the L11 protein of C. glutamicum.

As primer for production of the rplK deletion allele, the following oligonucleotides, which were prepared by ARK Scientific (ARK Scientific GmbH Biosystems, Darmstadt, Germany), were used in addition to the primers P1 up and P2 down described in Example 3.

| | |
|---|---|
| P1 down: | |
| 5′-Extension-CGC CGT GAG C-5′-side-GCC AAC TGG AGG AGC AGG GT-3′ | (see also SEQ ID No. 7) |
| P2 up: | |
| 540 -Extension-TCC AGT TGG C-5′-side-GCT CAC GGC GTC AAC ATC AG-3′: | (see also SEQ ID No. 8) |

The primers used for PCR were derived from the known DNA sequence. This has in each case a 10 bp 5′ extension, which is exactly complementary to the 5′ sides of the primers P1 up and P2 down. Chromosomal DNA was extracted from C. glutamicum ATCC 13032 and two 600 bp DNA fragments were first produced in separate PCR reactions with it as matrix using the given oligonucleotides P1 up, P1 down, P2 up and P2 down, the Pfu DNA polymerase (Stratagene, La Jolla, USA) and the PTC 100 thermocycler (MJ Research Inc., Waltham, USA). A cycle consisting of thermal denaturing (94° C., 90 sec), annealing (58° C., 90 sec) and the polymerase reaction (72° C., 90 sec) was carried out 35 times in each of these PCR reactions.

The first 600 bp DNA amplificate, designated as rplK part 1, was obtained with the oligonucleotides P1 up and P1 down. It contains the 5′ region of the rplK gene (nucleotide 1-87) and additionally the 10 bp extension deriving from oligonucleotide P1 down, which corresponds to the nucleotides 100-109 of the rplK gene (SEQ ID No. 1). Thus this amplified rplK gene region has a 12 bp gap compared to the chromosomal DNA template that was used. The second 600 bp DNA fragment, rplK part 2, was obtained with the oligonucleotides P2 down and P2 up and contains the 3′ region of the rplK gene (nucleotide 78-435) and carries the identical gap (nucleotide 88-99) within the amplified rplK gene region. These two 600 bp DNA amplificates accordingly have a 20 bp overlapping DNA region. The two 600 bp PCR products rplK part 1 and rplK part 2 were now used in an additional PCR reaction together as DNA template, where the leading strand of rplK part 1 could be bonded to the lagging strand of rplK part 2 because of the overlapping complementary DNA region. The extension of this overlapping DNA region or the addition of the oligonucleotides P1 up and P2 down led to the formation of a 1200 bp PCR amplificate, which contains a 12 bp deletion derivative of the C. glutamicum rplK gene. A cycle consisting of thermal denaturing (94° C., 90 sec), annealing (58° C., 90 sec) and the polymerase reaction (72° C., 90 sec) was carried out 35 times in each of these PCR reactions.

The nucleotide sequence of the ΔrplK allele is represented in SEQ ID No. 3 and the variation of the L11 protein coded by this allele is represented in SEQ ID No. 4. This L11 protein variation lacks the tetrapeptide proline-alanine-leucine-glycine corresponding to the amino acid positions 30 to 33 of the wild type form of the L11 protein represented in SEQ ID No. 2.

### Example 5

Insertion of the ΔrplK Allele into the Chromosome

The ΔrplK allele, which contains a 12 bp deletion in the rplK gene, was inserted into the chromosome of C. glutamicum by means of integration mutagenesis with the help of the sacB system described in Schäfer et al., Gene, 14, 69-73 (1994) I.B.R. This system enables the specialist to make the identification or selection of allele exchanges that are executed through homologous recombination.

1. Construction of the Exchange Vector pΔrplK

The 1200 bp rplK deletion allele ΔrplK obtained in Example 4 was purified by means of the Qiagen PCR purification spin kit (Qiagen, Hilden, Germany) and used for ligation with the mobilizable cloning vector pK18mobsacB described in Schäfer et al., Gene, 14, 69-73 (1994) I.B.R. This vector was linearized beforehand with the restriction enzyme SmaI (Amersham-Pharmacia, Freiburg, Germany), mixed with the rplK deletion allele and treated with T4 DNA ligase (Amersham-Pharmacia, Freiburg, Germany).

The result was the plasmid pΔrplK.

Transformation of the E. coli strain DH5α with the plasmid pΔrplK took place as described in Tauch et al., FEMS Microbiological Letters, 123, 343-347 (1994) I.B.R. The transformants were selected on LBG agar, to which kanamycin (Merck, Darmstadt, Germany) (50 mg/L) had been added. The strain DH5α/pΔrplK was obtained in this way.

A clone was selected and characterized as ATCC13032ΔrplK.

2. Conduct of the Allele Exchange

A chromosomal 12 bp deletion in the rplK gene of C. glutamicum was obtained by means of integration mutagenesis using the sacB system described in Schäfer et al., Gene, 14, 69-73 (1994) I.B.R. This system allows the specialist to make the identification or selection of allele exchanges that are executed by homologous recombination.

The mobilizable plasmid pΔrplK was then inserted in the strain C. glutamicum ATCC 13032 as recipient starting from the E. coli donor strain S17-1 described in Simon et al., Bio/Technology, 1, 784-794 (1993) I.B.R. using the conjugation method described by Schäfer et al., Journal of Bacteriology, 172, 1663-1666 (1990) I.B.R. Since the plasmid pΔrplK cannot replicate in C. glutamicum, establishing it is possible only by integration into the C. glutamicum chromosome via homologous recombination between the plasmid-coded rplK deletion fragment and the identical chromosomal rplK gene region. The transconjugants were selected on LBG agar, to which kanamycin (25 mg/L) (Merck, Darmstadt, Germany) and nalidixic acid (Merck, Darmstadt, Germany) (50 mg/L) had been added.

Selection on the subsequent excision of the plasmid pΔrplK with the aid of the sacB system could be carried out only using a wild type allele of rplK. For this the plasmid prplK constructed in Example 3, which carries the complete rplK gene, was transferred into the integrant strain by electroporation by the method of Liebl et al., FEMS Microbiology Letters 65, 299-304 (1989) I.B.R. Selection of the strain took place on LBG agar, to which kanamycin (25 mg/L) (Merck, Darmstadt, Germany) and chloramphenicol (10 mg/L) (Merck, Darmstadt, Germany) had been added.

A selected transformed colony was transinoculated in 100 mL LBG liquid medium (in a 250 mL Erlenmeyer flask with baffles) and incubated for 24 h at 30° C. and 300 rpm. Then 2×10⁶ cell/mL of this liquid culture was applied to LBG agar that contained 10% sucrose (Merck, Darmstadt, Germany) and incubated for 48 h at 30° C. C. glutamicum cells that were capable of growing on this medium had lost the integrated plasmid pΔrplK as a consequence of a second recombination event between the rplK deletion allele and the natural rplK region. This second recombination event leads either to reformation of the natural chromosomal rplK gene arrangement or it results in the generation of a C. glutamicum pΔrplK mutant in which the 12 bp N-terminal DNA fragment is missing. The chromosomal DNA was extracted from the selected “sucrose-resistant” and potential pΔrplK-bearing C. glutamicum cells. This served as matrix with which the oligonucleotides Pdel up and Pdel down2 were derived using the rplK sequence (ARK Scientific GmbH Biosystems, Darmstadt, Germany). The PCR experiments were carried out using the primers, Pfu DNA polymerase (Stratagene, La Jolla, USA) and the PTC 100 thermocycler (MJ Research Inc., Waltham, USA). A cycle consisting of thermal denaturing (94° C., 90 sec), annealing (58° C., 90 sec) and the polymerase reaction (72° C., 90 sec) was carried out 35 times in the PCR. Then the resulting DNA amplificates were purified by means of the Qiagen PCR purification spin kit (Qiagen, Hilden, Germany). Analysis of the nucleotide sequences of the purified DNA amplificates, which was carried out as described above, showed that in 43% of the cases a DNA amplificate had been produced that lacked the 12 bp DNA region. Accordingly, in the relevant pΔrplK-bearing transconjugants the second recombination event had led to the formation of the chromosomal 12 bp deletion in the rplK gene.

Then the plasmid prplK was removed from a selected deletion-bearing transconjugant by the plasmid curing method described in Schäfer et al., Journal of Bacteriology, 176, 7309-7319 (1994) I.B.R. The resulting strain of C. glutamicum ATCC13032 ΔrplK thus carries a chromosomal 12 bp deletion within the rplK gene, which leads to the loss of the tetrapeptide proline-alanine-leucine-glycine of the L11 protein.

### Example 6

Preparation of Lysine

The strain C. glutamicum ATCC13032 ΔrplK obtained in Example 5 was cultured in a nutrient medium suitable for production of lysine and the lysine content in the culture supernatant was determined.

For this the strain was first incubated on agar plates (brain-heart agar) for 24 h at 33° C. Starting from these agar plate cultures a preculture was inoculated (10 mL medium in 100 mL Erlenmeyer flasks). The complete medium CgIII (10 g/L bactopeptone, 10 g/L yeast extract, 2.5 g/L NaCl, 20 g/L glucose, pH 7.4) was used as medium for the preculture. The preculture was incubated for 24 h at 130° C. and 240 rpm on the shaker. A primary culture was inoculated from this preculture, so that the starting optical density (660 nm) of the main culture was 0.1 OD. The medium MM was used for the main culture.

Medium MM

| | | |
|---|---|---|
| CSL (corn steep liquor) | 5 | g/L |
| MOPS (morpholinopropane sulfonic acid) | 20 | g/L |
| Glucose (separately.autoclaved) | 50 | g/L |
| (NH₄)₂(SO₄) | 25 | g/L |
| KH₂PO₄ | 0.1 | g/L |
| MgSO₄ ● 7 H₂O | 1.0 | g/L |
| CaCl₂ ● 2 H₂O | 10 | mg/L |
| FeSO₄O ● 7 H₂O | 10 | mg/L |
| MnSO₄ ● H₂O | 5.0 | mg/L |
| Biotin (sterile filtered) | 0.3 | mg/L |
| Thiamine ● HCl (sterile filtered) | 0.2 | mg/L |
| CaCO₃ | 25 | g/L |

The CSL, MOPS and salt solution are adjusted to pH 7 with ammonia water and autoclaved. Then the sterile substrate and vitamin solutions are added, as well as the dry autoclaved CaCO₃.

Culturing takes place in 10 mL volume in a 100 mL Erlenmeyer flask with baffles. Culturing took place at 33° C. and 80% air humidity.

After 48 h the OD at a measurement wavelength of 660 nm was determined with the Biomek 1000 (Beckmann Instruments GmbH, Munich). The amount of lysine that formed was determined by means of an amino acid analyzer from the Eppendorf-BioTronik Company (Hamburg, Germany) by ion exchange chromatography and subsequent column derivatization with ninhydrin detection.

The result of the experiment is shown in Table 1.

TABLE 1

| Strain | OD (660) | Lysine ● HCl g/L |
|---|---|---|
| ATCC13032 ΔrplK | 13.0 | 0.98 |
| ATCC13032 | 13.8 | 0.0 |

It is understood that the foregoing detailed description is given merely by way of illustration and that many variations may be made therein without departing from the spirit of this invention and are intended to be encompassed by the claims appended hereto.

We claim:

1. An isolated polynucleotide containing a polynucleotide sequence selected from the group consisting of:
a) a polynucleotide that is at least 70% identical to a polynucleotide that codes for a polypeptide that contains the amino acid sequence of SEQ ID NO: 2,
b) a polynucleotide that codes for a polypeptide that contains an amino acid sequence that is at least 70% identical to the amino acid sequence of SEQ ID NO: 2,
c) a polynucleotide that is complementary to the polynucleotides of (a) or (b), and
d) a polynucleotide containing at least 15 successive bases of the polynucleotide sequence of (a), (b) or (c).

2. A polynucleotide as in claim 1, wherein the polynucleotide is a replicable DNA.

3. A polynucleotide as in claim 2, wherein the polynucleotide is a recombinant DNA.

4. A polynucleotide as in claim 1, wherein the polynucleotide is an RNA.

5. A polynucleotide as in claim 2, containing the nucleic acid sequence of SEQ ID NO: 1.

6. A polynucleotide as in claim 2, containing a polynucleotide sequence which codes for a polypeptide containing the amino acid sequence of SEQ ID NO: 2.

7. A polynucleotide as in claim 1, containing the nucleotide sequence as represented in SEQ ID NO: 3.

8. A polynucleotide as in claim 1, containing at least 15 successive bases of the nucleotide sequence as represented in SEQ ID NO: 3.

9. A polynucleotide as in claim 1, containing a polynucleotide sequence which codes for a polypeptide containing at least 5 successive amino acids of the amino acid sequence represented in SEQ ID NO: 4.

10. Replicable DNA as in claim 2, containing
(i) the nucleotide sequence shown in SEQ ID NO: 1, or
(ii) at least one sequence that corresponds to the sequence (i) within the region of degeneration of the genetic code, or
(iii) at least one sequence that hybridizes with the complementary sequence to sequence (i) or (ii), and optionally
(iv) functionally accurate sense mutants in (i).

11. A vector containing a polynucleotide as in claim 1, wherein the polynucleotide is deposited in E. coli DH5α/pΔrplK as DSM 13158.

12. A vector as in claim 11, wherein the polynucleotide contains the sequence of SEQ ID NO: 3.

13. Coryneform bacteria serving as host cells that contain a deletion or an insertion in the rplK gene, or cell lysate of such bacteria.

14. A method for preparation of an amino acid, comprising:
a) fermenting bacteria, in which at least the rplK gene is attenuated, to produce the amino acid, and
b) enriching the amino acid in a medium or in a cell of the bacteria.

15. The method as in claim 14, further comprising isolating said amino acid.

16. A method as in claim 14, wherein the amino acid is L-lysine.

17. A method as in claim 14, wherein, in the bacteria, additional genes of the biosynthesis pathway of the amino acid are enhanced.

18. A method as in claim 14, wherein, in the bacteria, metabolic pathways that reduce formation of the amino acid are at least partially turned off.

19. A method as in claim 14, wherein expression of a polynucleotide, in the bacteria, is reduced and said polynucleotide contains a polynucleotide sequence selected from the group consisting of:
a) a polynucleotide that is at least 70% identical to a polynucleotide that codes for a polypeptide that contains the amino acid sequence of SEQ ID NO: 2,
b) a polynucleotide that codes for a polypeptide that contains an amino acid sequence that is at least 70% identical to the amino acid sequence of SEQ ID NO: 2,
c) a polynucleotide that is complementary to the polynucleotides of (a) or (b), and
d) a polynucleotide containing at least 15 successive bases of the polynucleotide sequence of (a), (b) or (c).

20. A method as in claim 14, wherein a catalytic property of a polypeptide, in the bacteria, is reduced and the polypeptide is coded by a polynucleotide which contains a polynucleotide sequence selected from the group consisting of:
a) a polynucleotide that is at least 70% identical to a polynucleotide that codes for a polypeptide that contains the amino acid sequence of SEQ ID NO: 2,
b) a polynucleotide that codes for a polypeptide that contains an amino acid sequence that is at least 70% identical to the amino acid sequence of SEQ ID NO: 2,
c) a polynucleotide that is complementary to the polynucleotides of (a) or (b), and
d) a polynucleotide containing at least 15 successive bases of the polynucleotide sequence of (a), (b) or (c).

21. A method as in claim 14, wherein one uses bacteria in which an insertion mutagenesis is produced for attenuation, using the plasmid pΔrplK deposited as DSM 13158.

22. A method as in claim 14, wherein bacteria are fermented in which one or more of the following genes is overexpressed, said one or more genes is selected from the group consisting of:
a) the dapA gene coding for dihydrodipicolinate synthase,
b) a feedback resistant aspartate kinase,
c) the DNA fragment mediating S-(2-aminoethyl) cysteine resistance,
d) the pyc gene coding for pyruvate carboxylase,
e) the mqo gene coding for malate: quinone oxidoreductase,
f) the lysE gene coding for lysine export, and
g) the zwa1 gene.

23. A method as in claim 14, wherein bacteria are fermented in which one or more of the following genes is attenuated, said one or more genes is selected from the group consisting of:
a) the pck gene coding for phosphoenol pyruvate carboxykinase,
b) the pgi gene coding for glucose 6-phosphate isomerase,
c) the poxB gene coding for pyruvate oxidase,
d) the zwa2 gene, and
e) the rela gene coding for the PPGPP synthetase I.

24. A method as in claim 14, wherein the bacteria is a microorganism of the family Corynebacterium glutamicum.

25. A method of using a polynucleotide sequence as in claim 1, as a primer for preparation of the DNA of genes that lack action corresponding to the rplK gene, via the polymerase chain reaction.

26. A method of using a polynucleotide sequence as in claim 1, as a hybridization probe.

27. Bacteria, in which at least the rplK gene is modified to enhance production of an ammo acid.

28. Bacteria, according to claim 27, wherein said bacteria are fermented.

29. A composition comprising bacteria in which at least the rplK gene is modified to enhance production of an amino acid.

30. A composition according to claim 29, in which the bacteria are living.

31. A composition according to claim 29, in which the bacteria are dead.

32. A composition according to claim 29, in which the bacteria are Coryneform bacteria.


SEQUENCE LISTING (8 sequences)

SEQ ID NO: 1 - DNA, 835 bp, Corynebacterium glutamicum
  CDS (201)..(635)
ttgcgtgtagggtagacaatcgcgtgttttttaagcatgctcaaaatcattcatccccggtggcccggttacgtaaagatcagcaaagatgatcaactaaagcgatcatctgaagttgtagcgggaccgagcatccggacggttactagtggggtttcatcgtcccagttgtggccggtaacaaggaagcaggtttaacgatggctcctaagaagaagaagaaggtcactggcctcatcaagctccagatccaggcaggacaggcaaaccctgctcctccagttggcccagcacttggtgctcacggcgtcaacatcatggaattctgcaaggcttacaacgctgcgactgaaaaccagcgcggcaacgttgttcctgttgagatcaccgtttacgaagaccgttcattcgacttcaagctgaagactcctccagctgcaaagcttcttctgaaggctgctggcctgcagaagggctccggcgttcctcacacccagaaggtcggcaaggtttccatggctcaggttcgtgagatcgctgagaccaagaaggaagacctgaacgctcgcgatatcgacgctgctgcgaagatcatcgctggtaccgctcgttccatgggcatcaccgtcgaaggctaaaagctttcacaccggttagtggctcattcaaaatgaatggccaccaaccaattttcaccaaagttttatgtggcagggccagctccggcccgttaaaccacagaattccatgaaagggaatttctaatgagcaagaactctaaggcgtaccgcgaggccgctgagaagatcgacgctggtcgcatctactccccact

SEQ ID NO: 2 - protein, 145 aa, Corynebacterium glutamicum
MAPKKKKKVTGLIKLQIQAGQANPAPPVGPALGAHGVNIMEFCKAYNAATENQRGNVVPVEITVYEDRSFDFKLKTPPAAKLLLKAAGLQKGSGVPHTQKVGKVSMAQVREIAETKKEDLNARDIDAAAKIIAGTARSMGITVEG

SEQ ID NO: 3 - DNA, 825 bp, Corynebacterium glutamicum
  CDS (200)..(622)
tgcgtgtagggtagacaatcgcgtgttttttaagcatgctcaaaatcattcatccccggtggcccggttacgtaaagatcagcaaagatgatcaactaaagcgatcatctgaagttgtagcgggaccgagcatccggacggttactagtggggtttcatcgtcccagttgtggccggtaacaaggaagcaggtttaacgatggctcctaagaagaagaagaaggtcactggcctcatcaagctccagatccaggcaggacaggcaaaccctgctcctccagttggcgctcacggcgtcaacatcatggaattctgcaaggcttacaacgctgcgactgaaaaccagcgcggcaacgttgttcctgttgagatcaccgtttacgaagaccgttcattcgacttcaagctgaagactcctccagctgcaaagcttcttctgaaggctgctggcctgcagaagggctccggcgttcctcacacccagaaggtcggcaaggtttccatggctcaggttcgtgagatcgctgagaccaagaaggaagacctgaacgctcgcgatatcgacgctgctgcgaagatcatcgctggtaccgctcgttccatgggcatcaccgtcgaaggctaaaagctttcacaccggttagtggctcattcaaaatgaatggccaccaaccaattttcaccaaagttttatgtggcagggccagctccggcccgttaaaccacagaattccatgaaagggaatttctaatgagcaagaactctaaggcgtaccgcgaggccgctgagaagatcgacgctggtcgcatctactccccactcga

SEQ ID NO: 4 - protein, 141 aa, Corynebacterium glutamicum
MAPKKKKKVTGLIKLQIQAGQANPAPPVGAHGVNIMEFCKAYNAATENQRGNVVPVEITVYEDRSFDFKLKTPPAAKLLLKAAGLQKGSGVPHTQKVGKVSMAQVREIAETKKEDLNARDIDAAAKIIAGTARSMGITVEG

SEQ ID NO: 5 - DNA, 20 bp, Corynebacterium glutamicum
aggagcaggctgttgtcacc

SEQ ID NO: 6 - DNA, 20 bp, Corynebacterium glutamicum
gcggatagctacgtcgatgg

SEQ ID NO: 7 - DNA, 30 bp, Corynebacterium glutamicum
cgccgtgagcgccaactggaggagcagggt

SEQ ID NO: 8 - DNA, 30 bp, Corynebacterium glutamicum
tccagttggcgctcacggcgtcaacatcag